(19) United States
(12) Patent Application Publication
(10) Pub. No.: US 2022/0102013 A1
(43) Pub. Date: Mar. 31, 2022

(54) MEDICAL SERVICE SUPPORT DEVICE AND MEDICAL SERVICE SUPPORT SYSTEM

(71) Applicant: Canon Medical Systems Corporation, Otawara-shi (JP)

(72) Inventor: Keita MITSUMORI, Nasushlobara (JP)

(73) Assignee: Canon Medical Systems Corporation, Otawara-shi (JP)

(21) Appl. No.: 17/447,825

(22) Filed: Sep. 16, 2021

(30) Foreign Application Priority Data
Sep. 25, 2020 (JP) 2020-161282

(51) Int. Cl. G16H 70/20; G16H 10/60

(52) U.S. Cl. CPC G16H 70/20; G16H 10/60

(57) ABSTRACT

According to one embodiment, a medical service support device includes processing circuitry configured to: acquire medical ontology and input data entered in a remarks field of a medical record in a natural language; generate analysis data of the input data by executing natural language processing upon the input data; generate structured data in which the medical ontology is associated with the analysis data; generate confirmation data that expresses the structured data in the natural language; and display the confirmation data on a display.

### CROSS-REFERENCE TO RELATED APPLICATIONS

This application is based upon and claims the benefit of priority from Japanese Patent Application No. 2020-161282, filed Sep. 25, 2020, the entire contents of which are incorporated herein by reference.

### FIELD

Embodiments described herein relate generally to a medical service support device and a medical service support system.

### BACKGROUND

In the medical field, various tests including sampling tests and physiological function tests are conducted upon a test subject. In the remarks field of an electronic chart or radiogram interpretation report, ordering (purpose) and results (test results, treatments, etc.) of various tests are described in a natural language.

The data of the electronic charts and interpretation reports written in a natural language is unstructured, and therefore these charts and reports are difficult to use and leverage. For instance, when a large number of electronic charts or interpretation reports are intended to be put into statistical use, a technique of quantifying each structured element and constructing a data set (i.e., graph database) expressing the connection between the elements cannot be applied as is to the electronic charts or interpretation reports containing unstructured data. If, however, the procedure for inputting data to an electronic chart or interpretation report is changed to one that can structure the data at the time of inputting, a change in the procedure for inputting data on an input application may make the user feel burdened. For these reasons, user-friendly developments have been desired for the inputting procedure on an electronic chart or interpretation report, with fewer changes from the current inputting procedure. The electronic chart or interpretation report is a mere example of a medical record of a patient kept by a doctor, and may be referred to as a “medical record”. The medical record may also be referred to as “clinical information” or “medical information”.

### BRIEF DESCRIPTION OF THE DRAWINGS

FIG. 1 is a diagram for showing an exemplary structure of a medical service support system according to the first embodiment.

FIG. 2 is a schematic diagram for showing an exemplary data structure of medical ontology.

FIG. 3 is a flowchart for showing an exemplary procedure of a report creation supporting process by a diagnosis support device according to the first embodiment.

FIG. 4 is a diagram for showing an exemplary report creation screen displayed in the report creation supporting process performed by the medical service support device according to the first embodiment.

FIG. 5 is a schematic diagram for showing an exemplary data structure of structured data.

FIG. 6 is a diagram for showing an exemplary confirmation screen displayed in the report creation supporting process performed by the medical service support device according to the first embodiment.

FIG. 7 is a flowchart for showing the procedure of a report creation supporting process performed by a medical service support device according to the first modification example of the first embodiment.

FIG. 8 is a diagram for showing an exemplary revision recommendation screen displayed in the report creation supporting process performed by the medical service support device according to the first modification example of the first embodiment.

FIG. 9 is a diagram for showing an exemplary structure of a medical service support system according to the second embodiment.

FIG. 10 is a flowchart for showing an exemplary procedure of the report creation supporting process performed by the medical service support device according to the second embodiment.

FIG. 11 is a diagram for showing an exemplary confirmation screen displayed in the report creation supporting process performed by the medical service support device according to the second embodiment.

FIG. 12 is a diagram for showing an exemplary confirmation screen displayed in the report creation supporting process performed by the medical service support device according to the second embodiment.

FIG. 13 is a diagram for showing an exemplary confirmation screen displayed in the report creation supporting process performed by the medical service support device according to the second embodiment.

### DETAILED DESCRIPTION

In general, according to one embodiment, a medical service support device includes processing circuitry configured to: acquire medical ontology and input data entered in a remarks field of a medical record in a natural language; generate analysis data of the input data by executing natural language processing upon the input data; generate structured data in which the medical ontology is associated with the analysis data; generate confirmation data that expresses the structured data in the natural language; and display the confirmation data on a display.

Embodiments of a medical information processing device will be described in detail below by referring to the drawings. In the following description, structural elements having substantially the same functions and configurations will be denoted by the same reference symbols, and the same explanation will be given only where necessary.

### First Embodiment

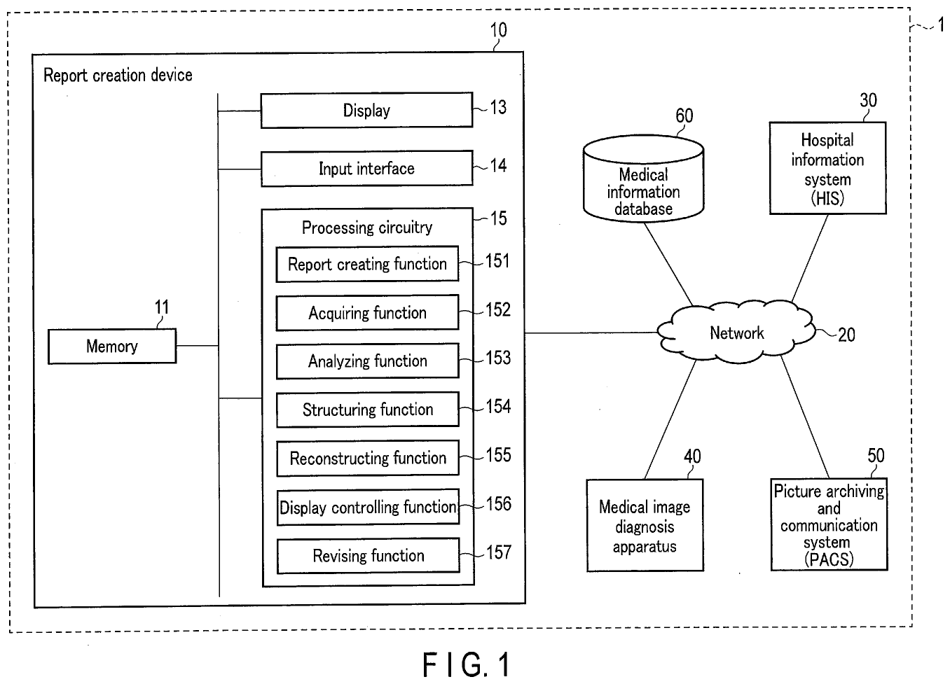

FIG. 1 is a diagram for showing the structure of a medical service support system 1 according to an embodiment. The medical service support system 1 includes a report creation device 10, which is configured to create medical records. The report creation device 10 is an example of the medical service support device.

A medical record contains a remarks field, in which a health-care worker's remarks on a patient and progress information of the patient are freely described. A medical record may include an interpretation report, electronic chart, and the like. A health-care worker may be a clinician who creates an electronic chart or a radiologist who creates an interpretation report. In the remarks field of the electronic chart, progress information of a patient is input in a natural language. The progress information of a patient includes, for example, symptoms, observation, a test list, test results, a treatment list, treatment results, and test ordering (purpose). In the remarks field of the interpretation report, observations and impressions of medical images generated by a medical image diagnosis apparatus 40 are input in a natural language.

The report creation device 10 is coupled through a network 20 to a hospital information system (hereinafter referred to as “HIS”) 30, a medical image diagnosis apparatus 40, a picture archiving and communication system (hereinafter referred to as “PACS”) 50, and a medical information database 60.

The network 20 may be a local area network (LAN). The connection to the network 20 may be established in either a wired or wireless manner. In addition, the connection is not limited to LAN if security can be ensured by a virtual private network (VPN) or the like. A connection to the

Internet or other public communication network may be adopted.

The HIS 30 is configured to manage information relating to a medical facility of the hospital or the like. The medical facility-related information includes patient information, test information, and the like.

The medical image diagnosis apparatus 40 may be an X-ray computed tomographic apparatus, a magnetic resonance imaging apparatus, an X-ray diagnostic apparatus or the like.

The PACS 50 is configured to store medical images output from the medical image diagnosis apparatus 40, interpretation reports created based on the medical images, and the like.

The medical information database 60 is configured to store a medical ontology. The medical ontology is a database for storing the relationship among medical care-related languages and between medical care-related languages and medical care-related classification (hereinafter referred to as “medical classification”). The medical ontology may be referred to as “clinical ontology”. The medical information database 60 is an example of an ontology storage unit.

The medical classification is used for identifying the medical meaning of medical care-related language (hereinafter referred to as “medical language”). On an electronic chart, the medical classification may include items of “subjective data (Subject)”, “objective data (Object)”, “diagnosis (Assessment)”, and “critical path (Plan)”. On an interpretation report, the medical classification may include items of “introduction”, “development”, “consideration”, and “conclusion”.

The medical ontology includes multiple nodes. Each node has score information. The score information may include scores indicating the degree of association among the nodes (hereinafter referred to as “inter-node scores), and scores indicating the degree of association between the nodes and medical classification (hereinafter referred to as “classification scores”). An inter-node score may indicate a value of a frequency of a certain medical language modifying another medical language. A classification score may indicate a value for a frequency of a certain medical language classified into a certain classification item. The inter-node score and classification score may change depending on a combination of other neighboring medical languages of the certain medical language.

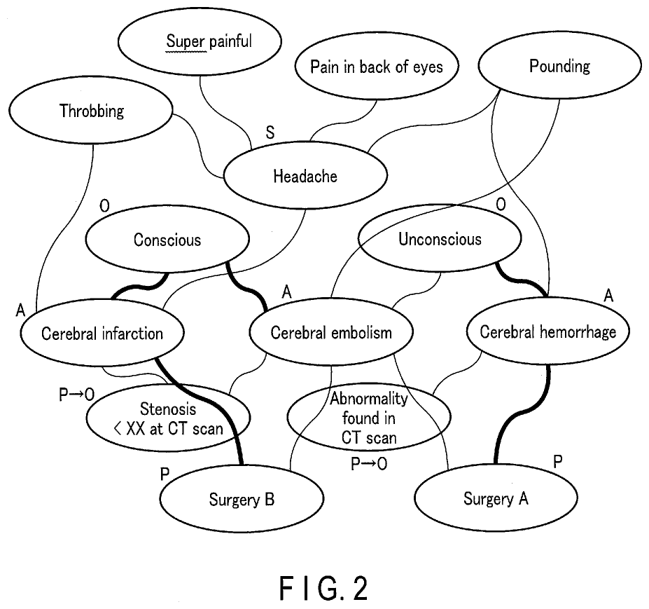

FIG. 2 is a schematic diagram for showing an exemplary data structure of medical ontology. The medical ontology includes medical languages as nodes. In the medical ontology, the nodes with high degrees of association are connected to each other. A node and an inter-node connection are associated with an item of the medical classification. For instance, in FIG. 2, the node “headache” is associated with a classification item “subjective data (Subject)”, and the node “cerebral infarction” is associated with a classification item “consideration (Assessment)”. The node “conscious” is associated with a classification item “objective data (Object)”, and the node “surgery A” is associated with a classification item “critical path (Plan)”.

The report creation device 10 exchanges various types of information with connected destinations through the network 20. The report creation device 10 creates a medical record based on the received information and data input by the health-care worker. If the medical record is an electronic chart, the report creation device 10 outputs the created electronic chart to the HIS 30. If the medical record is an interpretation report, the report creation device 10 outputs to the PACS 50 the created interpretation report in association with the medical images that have been referred to at the time of creating the interpretation report.

The report creation device 10 is provided with a memory 11, a display 13, an input interface 14, and a processing circuitry 15. In the description below, the report creation device 10 is assumed to execute various functions with a single console. These functions, however, may be executed with different consoles. For instance, the functions executed by the report creation device 10 may be provided in different console devices in a distributed manner.

The memory 11 is a storage device configured to store various types of information such as a hard disk drive (HDD), a solid state drive (SSD), and an integrated circuit. In addition to an HDD and SSD, the memory 11 may be a portable storage medium such as a compact disc (CD), a digital versatile disc (DVD), and a flash memory. The memory 11 may be a driving device for reading and writing various types of information from and to a semiconductor memory element such as a flash memory and a random access memory (RAM). The storage region of the memory 11 may be provided in the report creation device 10, or in an external storage device connected to the report creation device 10 by way of a network.

The memory 11 is configured to store, for example, medical information, programs to be executed by the processing circuitry 15, and various types of data to be used in the process performed by the processing circuitry 15. The memory 11 is an example of a storage unit. The medical information is information about a patient which is used in medical practice. The medical information may be linguistic element information, text information, image information, or non-image information generated based on the image information. The program may be installed in a computer through a network or from a non-transitory computer-readable storage medium to cause the computer to realize the functions of the processing circuitry 15. In general, the data discussed throughout this specification is digital data.

The display 13 is configured to display various types of information. For instance, the display 13 outputs medical information generated by the processing circuitry 15, a graphical user interface (GUI) for receiving various operations from the operator, and the like. The display 13 may be a liquid crystal display or a cathode-ray tube (CRT) display. The display 13 may display a screen for creating an electronic chart that contains a remarks field, or a chart summary, which will be described later. The display 13 is an example of a display unit.

The input interface 14 is configured to receive various types of input operations from the operator, convert the received input operations to electrical signals, and output the signals to the processing circuitry 15. For instance, the input interface 14 receives input of medical information and input of various command signals from the operator. The input interface 14 is realized by a mouse, a keyboard, a trackball, switch buttons, a touch screen in which a display screen and a touch pad are integrated, a non-contact input circuit adopting optical sensors, a voice input circuit, and the like for performing various processes of the processing circuitry 15. The input interface 14 is connected to the processing circuitry 15 so that the input operation received from the operator can be converted to an electrical signal and output to the control circuit. Throughout this specification, the input interface is not limited to a physically operating component such as a mouse and a keyboard. Examples of the input interface include electrical signal processing circuitry configured to receive an electrical signal corresponding to an input operation from a separately provided external input device, and output this electrical signal to the processing circuitry 15. The input interface 14 is an example of an input unit.

The processing circuitry 15 is configured to control the overall operation of the report creation device 10. The processing circuitry 15 is a processor configured to, when calling up the program in the memory 11, realize a report creating function 151, an acquiring function 152, an analyzing function 153, a structuring function 154, a reconstructing function 155, and a revising function 157.

In FIG. 1, a single processing circuitry 15 is explained as realizing the report creating function 151, acquiring function 152, analyzing function 153, structuring function 154, reconstructing function 155, display controlling function 156 and revising function 157. The processing circuitry 15, however, may be such that multiple independent processors are combined to form processing circuitry in which the functions are realized when each processor executes a program. The report creating function 151, acquiring function 152, analyzing function 153, structuring function 154, reconstructing function 155, display controlling function 156, and revising function 157 may be referred to as a report creating circuit, an acquiring circuit, an analyzing circuit, a structuring circuit, a reconstructing circuit, a display controlling circuit, and a revising circuit, respectively, so that they can be mounted as separate hardware circuits. The same functions are executed by the processing circuitry 15 in the embodiments and modification examples below.

The word “processor” used in the above explanation may be, for example, a central processing unit (CPU) or a graphics processing unit (GPU), or a circuit such as ASIC, a programmable logic device (e.g., a simple programmable logic device (SPLD) and a complex programmable logic device (CPLD)), and a field programmable gate array (FPGA). The processor realizes functions by reading and executing the program stored in the memory 11. Instead of storing the program in the memory 11, the program may be directly incorporated into the circuit of the processor. If this is the case, the processor realizes the functions by reading and executing the program incorporated in the processor. The processors according to the embodiments are not limited to a single circuit for each processor, but may be configured as a process by combining different independent circuits to realize the functions. Furthermore, the structural components illustrated in FIG. 1 may be integrated into one processor to realize their functions. The above description of the “processor” applies to other embodiments and modification examples.

The processing circuitry 15 creates a medical record containing a remarks field with the report creating function 151. In particular, the processing circuitry 15 generates a GUI for inputting necessary items to the medical record, and displays the generated GUI (hereinafter also referred to as a “report creation screen”) on the display 13. If the medical record is an electronic chart, a GUI for creating an electronic chart is displayed on the display 13. If the medical record is an interpretation report, a GUI for creating an interpretation report is displayed on the display 13. An interpretation target medical image may be displayed on the GUI for creating an interpretation report. The processing circuitry 15 creates a medical record in accordance with the input data entered on the report creation screen. The processing circuitry 15 that realizes the report creating function 151 is an example of a report creating unit.

The processing circuitry 15 acquires, with the acquiring function 152, text data (hereinafter referred to as “input data”) input to the remarks field of the medical record in a natural language and medical ontology. Here, the processing circuitry 15 acquires the medical ontology from the medical information database 60 through the network 20. The processing circuitry 15 that realizes the acquiring function 152 is an example of an acquiring unit.

The processing circuitry 15 performs, with the analyzing function 153, natural language processing (NLP) upon the input data, thereby generating analysis data of the input data. For instance, the processing circuitry 15 executes a morphological analysis upon the input data to divide the input data of the natural language into multiple linguistic elements, and stores the divided linguistic elements as analysis data. The processing circuitry 15 that realizes the analyzing function 153 is an example of an analyzing unit.

The processing circuitry 15 generates, with the structuring function 154, data (hereinafter referred to as “structured data”) in which the analysis data is associated with the medical ontology. Here, the linguistic elements divided by NLP are connected with the nodes of the medical ontology. Using the medical ontology, the linguistic elements divided by NLP are individually connected to medical meanings. The structured data is generated by the NLP-automatically extracted analysis data tracking the connections of the existing medical ontology. The processing circuitry 15 that realizes the structuring function 154 is an example of the first generating unit.

The processing circuitry 15 generates, with the reconstructing function 155, text data (hereinafter referred to as “confirmation data”), by describing the structured data in a natural language. For instance, the processing circuitry 15 generates confirmation data by reconstructing the structured data, which has been obtained by structuring the input data, into text data in a natural language. The processing circuitry 15 that realizes the reconstructing function 155 is an example of the second generating unit.

The processing circuitry 15 controls the GUI to be displayed on the display 13 with the display controlling function 156. The processing circuitry 15 displays the confirmation data on the display 13 with the display controlling function 156. The processing circuitry 15 that realizes the display controlling function 156 is an example of a display controlling unit.

The processing circuitry 15, with the revising function 157, displays on the displaying unit a revision section to which a revision command is input to make a revision to the confirmation data, and revises the structured data based on the revision command input through the revision section. For instance, the processing circuitry 15 displays on the display 13 a GUI as a revision section for inputting a revision command to revise the display screen of the confirmation data, and revises the confirmation data and the structured data based on the revision command input through the revision section. With the user's revision, the connection of the structured data is changed, or a new connection of the structured data is established. The structured data to which the user's revision is reflected is stored in the medical information database 60, thereby updating the database storing the medical ontology. The processing circuitry 15 that realizes the revising function 157 is an example of a revising unit.

Next, the operation of the report creation supporting process executed by the report creation device 10 will be explained. In the report creation supporting process, the input data entered into the remarks field of the medical record in a natural language is divided into multiple linguistic elements using the NLP and medical ontology; the generated linguistic elements are individually connected with the nodes of the medical ontology; and the confirmation screen on which the linguistic elements are individually connected with the medical ontology is displayed, in order to assist the user in creating a medical record. According to the present embodiment, the confirmation data is displayed on the display 13 when storing the medical record.

The procedure of the report creation supporting process explained below is a mere example, and the operations of the process can be suitably changed. Omission, replacement and addition of steps may be made to the processing procedure described below according to the embodiment.

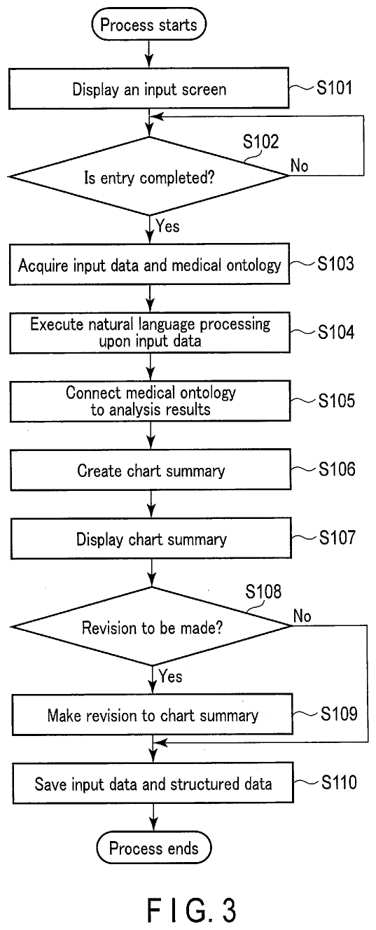

In the following example, the case of the medical record being an electronic chart will be explained. FIG. 3 is a flowchart for showing an exemplary procedure of the report creation supporting process according to the present embodiment. When inputting a patient's progress information into the remarks field of the electronic chart, a clinician may enter a command to begin the report creation supporting process on the input interface 14. Upon the entry of the command to begin the report creation supporting process on the input interface 14, the processing circuitry 15 initiates the report creation supporting process. The clinician is an example of an operator who inputs data into the medical record.

### (Report Creation Supporting Process)

### (Step S101)

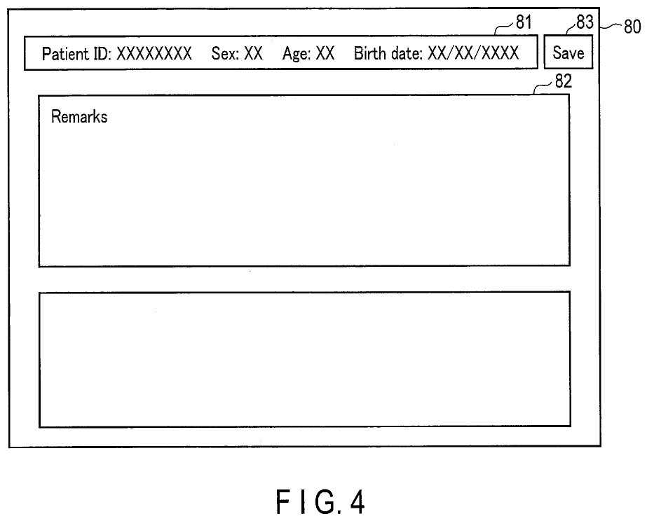

With the report creating function 151, the processing circuitry 15 generates a report creation screen 80, and displays the generated report creation screen 80 on the display 13. FIG. 4 shows an example of the report creation screen 80. The report creation screen 80 contains a patient information display portion 81, a remarks field 82, and a save button 83. The patient information display portion 81 shows patient information. The patient information may include a name, identification number, gender, age, birth date and the like. The patient information may be acquired from the HIS 30. In the remarks field 82, the clinician may enter the patient's progress information in a natural language. The clinician may use the input interface 14 such as a keyboard to enter the information in the remarks field 82. By way of the save button 83, the clinician enters an operation for storing the created electronic chart in association with the patient information.

### (Step S102)

With the report creating function 151, the processing circuitry 15 determines whether an entry in the remarks field 82 of the report creation screen 80 is completed. When the operation is entered with the save button 83, the processing circuitry 15 determines that the entry on the report creation screen 80 is completed (“Yes” at step S102). Then, the process proceeds to step S103.

### (Step S103)

With the acquiring function 152, the processing circuitry 15 acquires input data entered in the remarks field 82 and the medical ontology. For instance, the input data may be freely described text data such as “conscious; tingling in back of head; blood clot found in the past→cerebral infarction possible→diagnostic head imaging by MRI”. The “freely described” text may or may not fall into a certain classification item. The medical ontology may be acquired from the medical information database 60.

### (Step S104)

With the analyzing function 153, the processing circuitry 15 executes NLP upon the input data. The input data entered in a natural language is divided through the NLP into multiple linguistic elements. The processing circuitry 15 acquires the generated linguistic elements as analysis results.

### (Step S105)

With the structuring function 154, the processing circuitry 15 extracts from the medical ontology a node related to each of the linguistic elements generated by the NLP. The processing circuitry 15 associates each of the linguistic elements with the extracted node. One or more nodes may be associated with a linguistic element. If there is no node to be associated in the medical ontology, the linguistic element is left with no node associated with it. Next, the processing circuitry 15 acquires score information of the node (hereinafter referred to as “associated node”) associated with the data of each linguistic element. The score information contains an inter-node score and a classification score. The processing circuitry 15 associates the data of each linguistic element with the inter-node score and classification score of the associated node. The processing circuitry 15 stores, as structured data, the information including the linguistic elements as well as the associated nodes and score information that are individually associated with the linguistic elements.

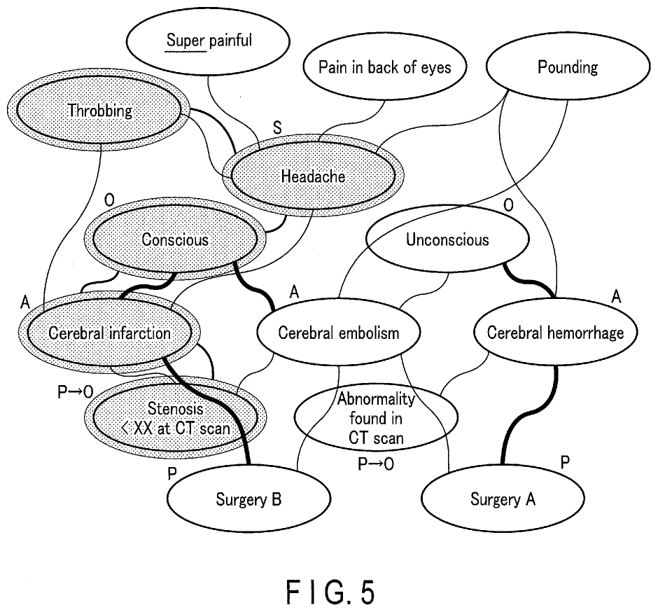

For instance, when the input data includes “tingling pain in back of the head”, the NLP generates linguistic elements, “head”, “back of head”, “pain”, “tingling” and the like. Thereafter, “head”, “headache”, and “tingling” are extracted from the medical ontology as associated nodes of the linguistic elements. Then, the score information of the extracted associated nodes is acquired. As the inter-node score of “throbbing”, the association scores with respect to the “head”, “eyes” and the like are acquired, and as the classification score, the association scores for different classification items are acquired. The acquired score information and information containing the associated nodes are associated for each linguistic element, so that the structured data can be generated. FIG. 5 is a schematic diagram for showing an exemplary data structure of the structured data. In the example of FIG. 5, “headache”, “throbbing”, “conscious”, “cerebral infarction”, and “stenosis <XX at CT scanning” are extracted as related nodes for the linguistic elements from among the nodes of the medical ontology. The structured data including the extracted nodes and score information between these nodes is thereby generated.

### (Step S106)

With the reconstructing function 155, the processing circuitry 15 generates, as confirmation data, text data (hereinafter referred to as a “chart summary”) by reconstructing in a natural language the structured data, which is obtained by structuring the input data entered into the electronic chart based on the linguistic elements and the score information associated with the linguistic elements. The linguistic elements are processed into a natural language, based on the associated nodes and the score information of the associated nodes. In the natural-language processing, a linguistic element associated with the classification scores of multiple classification items is connected, for instance, to a classification item having the highest association score among the associated classification scores.

### (Step S107)

With the display controlling function 156, the processing circuitry 15 displays the generated chart summary display screen 84 on the display 13. Furthermore, with the revising function 157, the processing circuitry 15 displays, as a revision section, the revision command input unit 86 on the chart summary display screen 84 of the display 13.

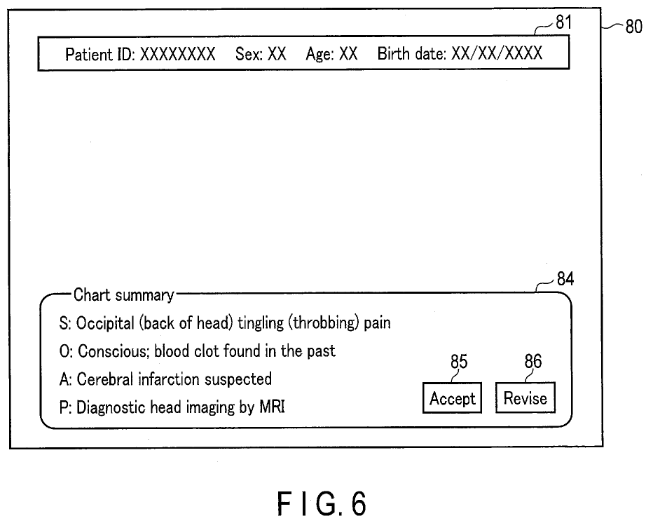

FIG. 6 shows an example of the chart summary display screen 84. In FIG. 6, the chart summary display screen 84 is superposed on the report creation screen 80. The chart summary display screen 84 shows a generated chart summary. For the classification items in the example of FIG. 6, “S”, representing the linguistic element being subjective data (Subject), “O”, representing the linguistic element being objective data (Object), “A”, representing the linguistic element being consideration (Assessment), and “P”, representing the linguistic element being a critical path (Plan), are adopted. In the example of FIG. 6, the input data “back of head”, “tingling”, “pain” and the like are associated with the classification item “S”. In addition, “tingling” of the input data is associated with an associated node “throbbing”. With the chart summary display screen 84 displayed on the display 13, the clinician looks at the displayed chart summary and determines whether or not the classification of the linguistic elements in the input data and the modification relation of the linguistic elements are appropriate.

The chart summary display screen 84 further shows a save command input unit 85 and a revision command input unit 86. The save command input unit 85 bears an indication “Accept”. The clinician inputs a command for saving the displayed chart summary on the save command input unit 85. The revision command input unit 86 bears an indication “Revise”. The clinician inputs a command to make a revision into the chart summary on the revision command input unit 86.

### (Step S108)

The processing circuitry 15 determines whether or not a revision will be made to the chart summary with the revising function 157. When a revision command is input on the revision command input unit 86 (“Yes” at step S108), the processing circuitry 15 determines that a revision will be made to the chart summary. In this case, the process proceeds to step S109. When a save command is input on the save command input unit 85 (“No” at step S108), the processing circuitry 15 determines that no revision will be made to the chart summary. In this case, the process proceeds to step S110.

### (Step S109)

When a revision command is input on the revision command input unit 86, the processing circuitry 15 generates a GUI (hereinafter referred to as a “chart summary revision screen”) for revising the chart summary, and displays this chart summary revision screen on the display 13. The clinician may select a revision target linguistic element on the chart summary revision screen, and thereby change the classification item associated with the selected linguistic element to another classification item. The clinician may also change the modification relation of the linguistic elements on the chart summary revision screen. In addition, the clinician may select a revision target linguistic element on the chart summary revision screen, and thereby change the node associated with this linguistic element to another node of the medical ontology.

### (Step S110)

When a save command is input on the save command input unit 85, the processing circuitry 15 stores the input data and the structured data corresponding to the current chart summary. Here, the processing circuitry 15 stores the structured data in association with the input data. The structured data and input data may be output to the HIS 30 or PACS 50.

As mentioned earlier, the medical ontology according to the present embodiment includes medical languages as nodes. Furthermore, certain nodes are connected to each other in the medical ontology. The input data is subjected to the natural language processing, through which analysis data is automatically extracted. Using the analysis data based on the connections in the medical ontology stored in the existing database, the structured data is generated. With the user's revision, the connection in the medical ontology may be changed, or a new connection may be established in the medical ontology. When the structured data to which a user's revision is reflected is saved, the database storing the medical ontology is updated.

The effects of the medical service support system 1 including a report creation device 10 according to the present embodiment will be described now.

The report creation device 10 according to the present embodiment is configured to acquire the medical ontology and the input data that has been entered in the remarks field of the medical record in a natural language, execute the natural language processing upon this input data to generate the analysis data of the input data, generate the structured data in which the analysis data is associated with the medical ontology, generate confirmation data in which the structured data is expressed in a natural language, and display the confirmation data on the display 13.

For instance, when the medical record is an electronic chart, the input data described in a natural language in the remarks field of the electronic chart is subjected to the NLP and thereby divided into multiple linguistic elements. Using the medical ontology, the structured data in which the NLP-divided linguistic elements are individually connected to the medical meanings can be generated. By creating a chart summary in which the linguistic elements connected to the medical meanings are expressed in a natural language based on the structured data, the structured data can be expressed in a text understandable for humans. In this manner, the person who has input the report checks the chart summary displayed on the display 13, and can thereby easily confirm whether each element of the input data entered into the electronic chart in a natural language is suitably associated with the medical ontology. That is, the person who has input the report can easily confirm whether the input data is suitably structured.

As described above, according to the present embodiment, the ease of use of a medical record containing a remarks field to which data is input in a natural language can be enhanced while minimizing the need to change the procedure for inputting data into a medical record. To be more specific, with no particular change to be made to the procedure for entering a description in the remarks field of the medical record, changes to the procedure for inputting data into the medical record can be minimized. In addition, structured data in which the analysis data of the input data in the remarks field is associated with the medical ontology is generated, and through this structured data, the ease of statistical use of the medical record can be enhanced. Moreover, by generating and displaying the confirmation data from the structured data, one can confirm that the structured data is suitably generated.

According to the present embodiment, the medical record may be referred to as an electronic chart or interpretation report. With regard to an electronic chart or interpretation report, the aforementioned effects can be produced.

The report creation device 10 according to the present embodiment displays on the display 13 a revision section for inputting a revision command to revise the confirmation data, and revises the structured data based on the revision command.

With the above structure, when the medical record is an electronic chart, even if the input data is not suitably structured, the creator of the electronic chart can easily revise the structured data by inputting a revision command using a revision section. As a result, the structured data based on the input data can be stored in a manner that further enhances the ease of use.

### (First Modification Example of First Embodiment)

The first modification example of the first embodiment will be described. In the modification example, the structure of the first embodiment is modified as indicated below. The same part of the structure, operations and effects as in the first embodiment will be omitted from the description.

According to the modification example, with the revising function 157, the processing circuitry 15 further acquires revision history information in which the history of revisions made to the confirmation data are recorded. Here, the processing circuitry 15 acquires the revision history information relating to the operator who has entered the data. The revision history information may include revisions previously made by this person, the number of revisions made in relation to a specific revision, and the frequency of revisions made in relation to the specific revision. Based on the revision history information, the processing circuitry 15 extracts items highly likely to be revised (hereinafter referred to as “revision candidate items”) from the input data, and displays the extracted revision candidate items on the display 13. The revision candidate items may be items having a number of revisions larger than a threshold value, or items having a frequency of revisions higher than a threshold value.

Next, the operations of the report creation supporting process executed by the report creation device 10 according to the modification example will be explained. According to the modification example, items of the input data that are highly likely to be revised are displayed on the display 13 at the time of inputting the input data.

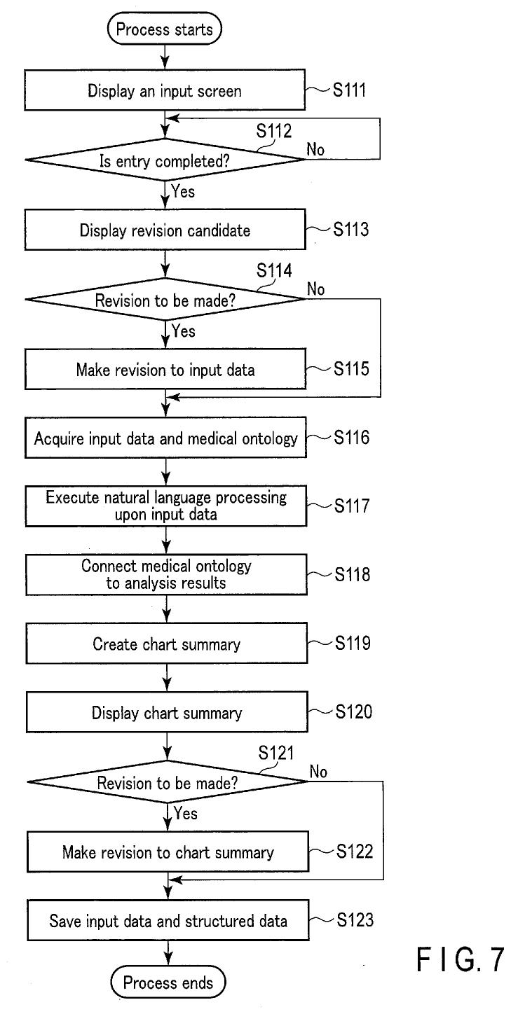

In the following example, the case of the medical record being an electronic chart will be explained. FIG. 7 is a flowchart for showing an exemplary procedure of the report creation supporting process according to the modification example. The operations at steps S111 to S112 and S116 to S123 are the same as the operations at steps S101 to S110 in FIG. 3, and the explanation thereof is omitted.

### (Report Creation Supporting Process)

### (Step S113)

When the operation of inputting data to the remarks field 82 of the report creation screen 80 is completed, the processing circuitry 15 acquires, with the revising function 157, the input data and the revision history information relating to the clinician. The revision history information may be stored in the HIS 30. The processing circuitry 15 extracts words included in the revision history information from the input data based on the revision history information. When the frequency of revisions in relation to the extracted words is a certain value or larger, the processing circuitry 15 determines this revision to be a revision candidate item. The processing circuitry 15 displays on the display 13 a GUI (hereinafter referred to as a “revision recommendation screen”) 87 through which revisions to the revision candidate items are recommended to the clinician.

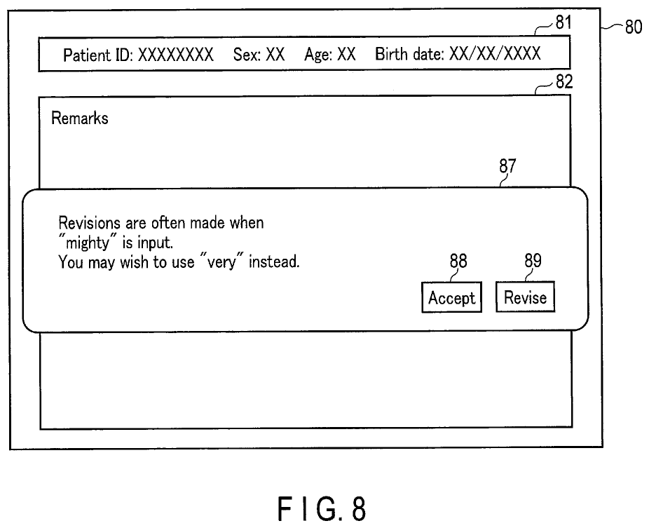

FIG. 8 shows an example of the revision recommendation screen 87. The revision recommendation screen 87 presents, to a clinician who frequently changes the word “mighty” to “very”, a sentence suggesting changing “mighty” that appears in the text input into the remarks field 82 to “very”. The revision recommendation screen 87 further includes a revision command non-inputting unit 88 and a revision command inputting unit 89. The revision command non-inputting unit 88 bears an indication “Accept”. On the revision command non-inputting unit 88, the clinician inputs a command to proceed with the process without making any revision. The revision command inputting unit 89 bears an indication “Revise”. On the revision command inputting unit 89, the clinician inputs a command to revise the input data.

### (Step S114)

With the revising function 157, the processing circuitry 15 determines whether or not the input data will be revised. If a revision command is input on the revision command inputting unit 89 (“Yes” at step S114), the process proceeds to step S115. If a save command is input on the revision command non-inputting unit 88 (“No” at step S114), the process proceeds to step S116.

### (Step S115)

If a revision command is input on the revision command inputting unit 89, the processing circuitry 15 revises the input data. Here, the processing circuitry 15 may display again the chart summary creation screen on the display 13 and have the clinician revise the input data. Alternatively, based on the revisions displayed on the revision recommendation screen 87, the revision to the input data may be automatically made.

In this modification example, the report creation device 10 extracts revision candidate items that are likely to be changed from the input data based on the revision history information, and displays the extracted items on the display 13. The operator who is entering the data can check the revision candidate items displayed on the display 13, which allows the operator to make a revision to a word likely to be revised later, at the time of inputting the data.

### Second Embodiment

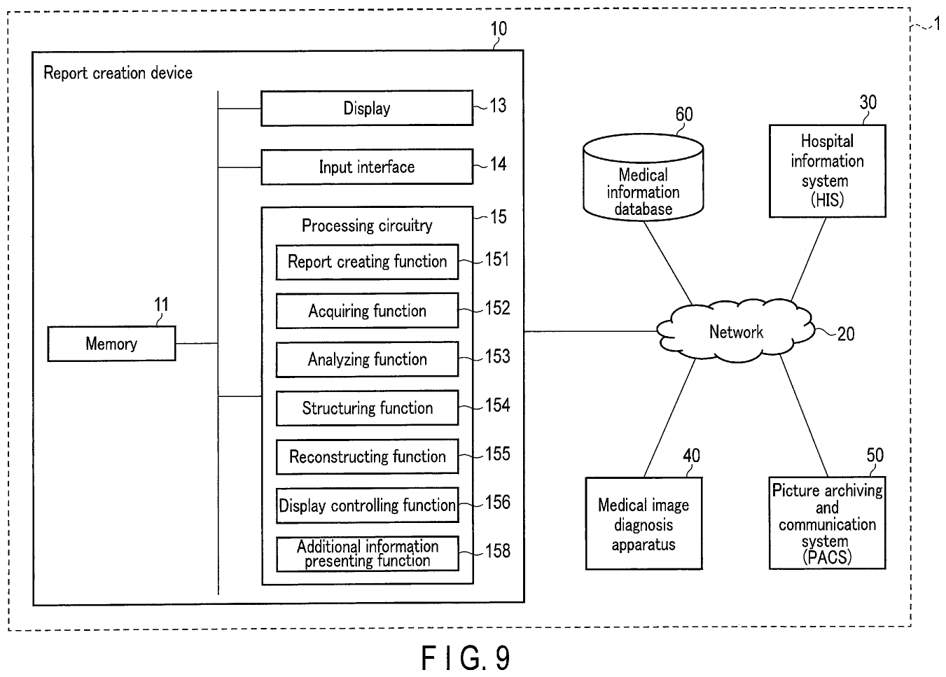

The second embodiment will be described. In the second embodiment, the structure of the first embodiment will be modified as indicated below. The same part of the structure, operations and effects as in the first embodiment will be omitted from the description. FIG. 9 shows the structure of the medical service support system 1 according to the second embodiment.

According to the embodiment, with the additional information presenting function 158, the processing circuitry 15 determines additional information (hereinafter referred to as “addition candidate information”) the input of which is recommended in addition to the input data, based on the input data and medical ontology, and displays the determined addition candidate information on the display 13. The addition candidate information may be results of specific tests, results of consultation regarding specific matters, information that is possibly an incomplete description, and the like. The processing circuitry 15 that realizes the additional information presenting function 158 is an example of an additional information presenting unit.

The processing circuitry 15 is further configured to, with the additional information presenting function 158, determine the order of displaying addition candidate information items on the display 13 based on the patient information. For instance, the order of displaying the addition candidate information items on the display 13 may be determined in accordance with the patient's age and gender.

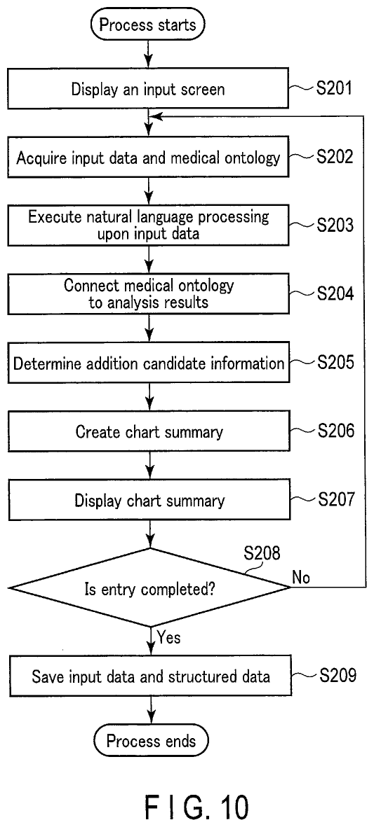

The operations of the report creation supporting process performed by the report creation device 10 according to the present embodiment will be described. In the following example, the case of the medical record being an electronic chart will be explained. FIG. 10 is a flowchart for showing an exemplary procedure of the report creation supporting process according to the present embodiment. The operations at steps S201, S203 to S204, S206, and S209 are the same as the operations at steps S101, S104 to S106, and S110 of FIG. 3, and the explanation thereof is omitted.

### (Report Creation Supporting Process)

### (Step S202)

With the acquiring function 152, the processing circuitry 15 acquires input data entered in the remarks field 82 and the medical ontology. Here, the processing circuitry 15 acquires the input data input into the remarks field 82 in real time, at certain time intervals. In this manner, every time an input is made into the remarks field 82, the processing circuitry 15 acquires the updated input data.

### (Step S205)

With the additional information presenting function 158, the processing circuitry 15 determines the addition candidate information based on the analysis data and medical ontology. Here, the processing circuitry 15 first acquires associated nodes associated with the linguistic elements generated through the NLP, and the inter-node scores of the respective associated nodes. Next, based on the inter-node scores, the processing circuitry 15 determines, for each of the associated nodes, whether or not the input data includes a node having an inter-node score larger than or equal to a certain value with respect to an associated node. The processing circuitry 15 determines that, if there is no node having an inter-node score larger than or equal to a certain value in the input data, the associated node has not yet established a connection with the medical ontology. The processing circuitry 15 thereby determines the addition candidate information, which is information required to establish a connection between the associated node determined to be not yet sufficiently connected with the medical ontology, and the medical ontology.

### (Step S207)

With the display controlling function 156, the processing circuitry 15 displays the generated chart summary display screen 84 on the display 13. Here, the processing circuitry 15 displays the chart summary display screen 84 within the report creation screen 80. The remarks field 82 and chart summary display screen 84 are therefore displayed together on the report creation screen 80. With the additional information presenting function 158, the processing circuitry 15 displays an addition candidate information displaying unit 90 indicating the addition candidate information within the report creation screen 80.

### (Step S208)

With the report creating function 151, the processing circuitry 15 determines whether the input into the remarks field 82 of the report creation screen 80 is completed.

For instance, when an operation is input with the save button 83, the processing circuitry 15 determines that the input into the report creation screen 80 is completed (“Yes” at step S208). Then, the process proceeds to step S209. The processing circuitry 15 repeats the operations of steps S202 to S207 until the input into the remarks field 82 of the report creation screen 80 is completed. In this manner, every time the input data in the remarks field 82 is updated, the structured data, chart summary and addition candidate information are updated.

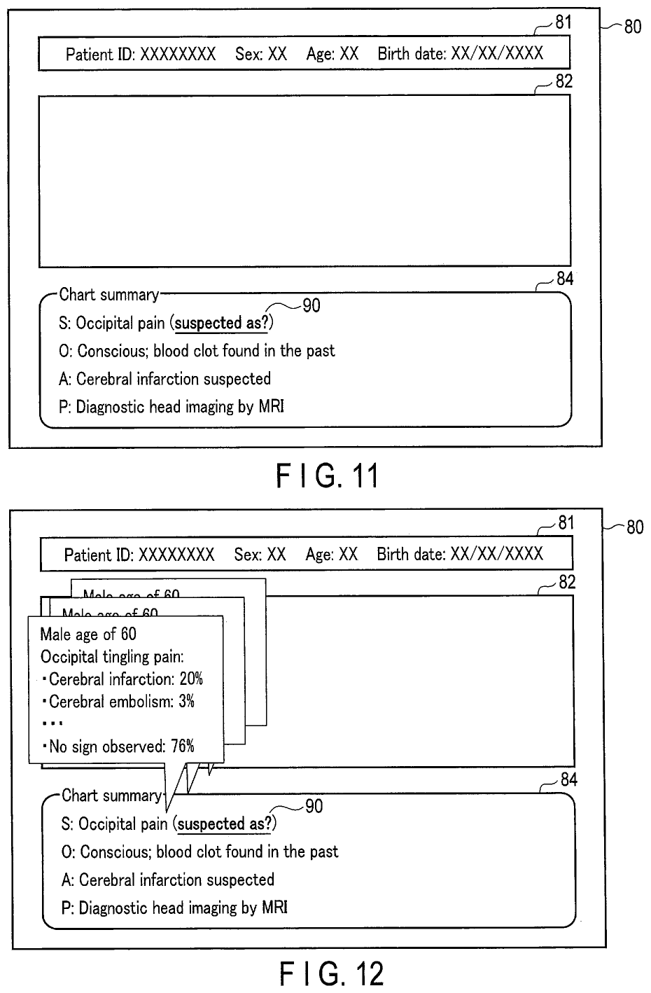

FIGS. 11 to 13 show an example of the report creation screen 80 according to the present embodiment. As illustrated in FIG. 11, the report creation screen 80 includes a chart summary display screen 84, in which an addition candidate information displaying unit 90 is displayed. The addition candidate information displaying unit 90 is presented for the expression “ache in head” on the chart summary display screen 84. The addition candidate information displaying unit 90 reads “suspected as?”. When the clinician selects the addition candidate information displaying unit 90, multiple input candidate display windows are displayed in the addition candidate information displaying unit 90 as illustrated in FIG. 12. In each of the input candidate display windows, the addition candidate information, names of diseases the patient may be afflicted with, the percentages of these diseases, and the like are displayed. In the input candidate display windows, the consultation results obtained through an additional consultation and the test results obtained through an additional test are displayed as addition candidate information. The input candidate display windows show different addition candidate information items. The input candidate display windows are mutually superposed on the screen. The input candidate display windows are placed from the front to the back of the screen in descending order of possibilities of obtaining the information through additional tests and consultations, based on statistical information in accordance with the patient's age and gender. When the clinician selects one of the input candidate display windows, the selected input candidate display window is displayed in an enlarged manner.

Based on the addition candidate information displayed on the input candidate display window, the clinician may conduct an additional test or consultation with the patient, and input the obtained test and consultation results into the remarks field 82. Thus, every time the input data in the remarks field 82 is updated, the chart summary displayed on the chart summary display screen 84 and the addition candidate information displayed on the addition candidate information displaying unit 90 are updated.

The input data entered in the remarks field 82, the chart summary displayed on the chart summary display screen 84, and the structured data may be automatically updated based on the addition candidate information corresponding to the input candidate display window selected by the clinician.

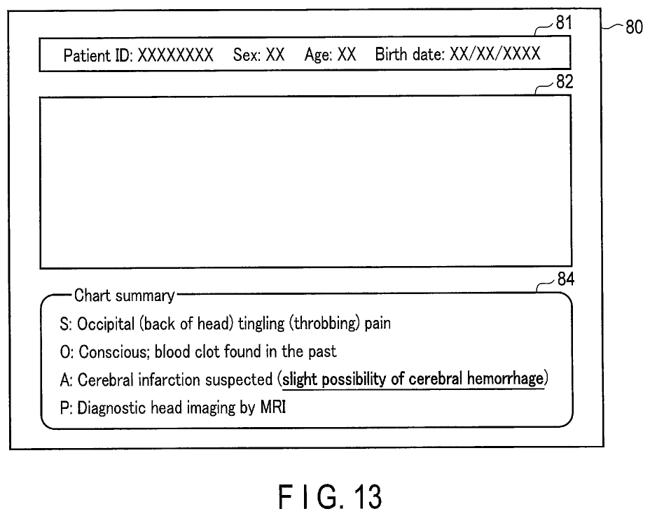

As illustrated in FIG. 13, information relating to an ailment not input into the remarks field 82 but with urgent attention required may be displayed on the display 13. In such a case, an ailment with urgent attention required is extracted from ailments suspected from the input data based on the input data and medical ontology, and the information relating to the extracted ailment is displayed on the chart summary display screen 84. In the example of FIG. 13, “cerebral hemorrhage” is extracted as an ailment with urgent attention required. In such a case, as a critical path for the “cerebral hemorrhage”, an indication recommending “CT image diagnosis” may be displayed on the chart summary display screen 84.

According to the present embodiment, the report creation device 10 determines, based on the input data and medical ontology, addition candidate information that is recommended as information to be added to the input data, and displays the determined addition candidate information on the display 13. The order of displaying the addition candidate information items is determined based on the patient information. The operator who has input the report checks the addition candidate information so as to confirm the list of tests to be additionally conducted, details of the consultation, and a presence/absence of incomplete descriptions. This enables the operator to create a highly reliable medical record.

According to at least one of the above embodiments, the ease of use of a medical record can be enhanced while minimizing changes to the procedure for inputting data into the medical record.

While certain embodiments have been described, these embodiments have been presented by way of example only, and are not intended to limit the scope of the inventions.

Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical service support device comprising processing circuitry configured to:
acquire medical ontology and input data entered in a remarks field of a medical record in a natural language;
generate analysis data of the input data by executing natural language processing upon the input data;
generate structured data in which the medical ontology is associated with the analysis data;
generate confirmation data that expresses the structured data in the natural language; and
display the confirmation data on a display.

2. The medical service support device according to claim 1, wherein
the medical record is either an electronic chart or an interpretation report.

3. The medical service support device according to claim 1, wherein
the processing circuitry is further configured to display a revision section to which a revision command for revising the confirmation data is entered on the display, and revise the confirmation data and the structured data in response to the revision command.

4. The medical service support device according to claim 3, wherein
the processing circuitry is further configured to extract, from the input data, a revision candidate item highly likely to be revised, based on revision history information, and display the revision candidate item on the display.

5. The medical service support device according to claim 1, wherein
the processing circuitry is further configured to determine addition candidate information to be added to the input data based on the input data and the medical ontology, and display the addition candidate information on the display.

6. The medical service support device according to claim 5, wherein
the processing circuitry is further configured to determine an order of displaying a plurality of items of 15. the addition candidate information based on patient information.

7. A medical service support system, comprising:
a storage medium configured to store medical ontology;
processing circuitry configured to
acquire the medical ontology and input data that is entered in a remarks field of a medical record in a natural language,
generate analysis data of the input data by executing natural language processing upon the input data,
generate structured data in which the medical ontology is associated with the analysis data,
generate confirmation data that expresses the structured data in a natural language; and
a display configured to display the confirmation data.
